(19) United States
(12) Patent Application Publication
(10) Pub. No.: US 2020/0138907 A1
(43) Pub. Date: May 7, 2020

(54) Composition for Use in the Treatment of an Individual Suffering a Condition Necessitating New Bone Formation

(71) Applicant: UMC Utrecht Holding B.V., Utrecht (NL)

(72) Inventors: Fetullah Cumhur Oner, Utrecht (NL); Jacqueline Alblas, Utrecht (NL); Michiel Croes, Utrecht (NL)

(21) Appl. No.: 16/334,555

(22) PCT Filed: Oct. 3, 2016
(86) PCT No.: PCT/EP2016/073551

§ 371 Date: Mar. 19, 2019

(51) Int. Cl. A61K 38/18; A61K 31/675; A61K 35/74; A61K 38/20; A61K 38/21; A61K 38/30; A61L 27/54; A61K 9/00

(52) U.S. Cl. CPC A61K 38/1875; A61K 31/675; A61K 35/74; A61K 38/20; A61K 45/06; A61K 38/30; A61L 27/54; A61K 9/0019; A61K 38/21

(57) ABSTRACT

Present invention relates to a composition for use in the treatment of an individual suffering from a condition necessitating new bone formation. The present invention further relates to osteoconductive carriers that have been provided with the composition of present invention for use in the treatment of an individual suffering a condition necessitating new bone formation. Also, present invention relates to a method for bone tissue generation in an individual suffering a condition necessitating new bone formation, such as large bone defects, segmental bone defects, structural bone defects, bone fractures, non-unions of bone fractures, fusion of joints including spinal fusions.

Present invention relates to a composition for use in the treatment of an individual suffering from a condition necessitating new bone formation. The present invention further relates to osteoconductive carriers that have been provided with the composition of present invention for use in the treatment of an individual suffering a condition necessitating new bone formation. Also, present invention relates to a method for bone tissue generation in an individual suffering a condition necessitating new bone formation, such as large bone defects, segmental bone defects, structural bone defects, bone fractures, non-unions of bone fractures, fusion of joints including spinal fusions.

Transplanting bone from the patient's own bone stock, an autologous bone graft, is the golden standard when it comes to the treatment of bone defects or fusion of joints. However, an autologous bone graft is not always an optimal solution for a patient, since bone must be harvested from the patient first, which results in a longer operation time and can also lead to pain and complications at the site where the bone is removed. Furthermore the autologous bone graft is not always available in sufficient quantities, nor always effective in patients. In a proportion of patients no fusion occurs with the use of autologous bone in spinal fusion or fracture non-unions.

Considering the relative high complication rate (10-40%) associated with autografts, the limited supply and unreliable effectivity of donor bone and the significant increase in surgery time associated with bone harvesting, new strategies are needed to reduce the widespread use of bone transplants for the regeneration and restoration of bone.

An alternative to the use of the patient's own bone is the use of donor bone (allogeneic bone, or demineralized bone matrix). These so called allografts are not as effective as autologous bone. One of the reasons is that the bone loses its bone-inductive abilities because this allogeneic bone must undergo treatment to minimize immunogenicity and risk of disease transmission.

In addition to the abovementioned bone transplantations, bone substitutes have been developed and approved to fill bone defects in numerous applications. These largely consist of calcium phosphates and calcium sulphates. Currently, there is no evidence supporting an osteoinductive action of these bone substitutes in vivo. As a result, these products can only support bone conduction in small defects.

Alternatively, to initiate new bone formation in surgical sites, bone-inductive growth factors such as bone morphogenetic proteins (BMPs) are available. Recombinant human

BMPs are used in orthopedic applications to induce new bone formation such as spinal fusions, non-unions or fractures. Furthermore, recombinant human BMPs are used in maxillofacial surgery, for instance in sinus lift surgery. BMPs are delivered to the surgical site by being incorporated into a carrier, and released to initiate new bone formation.

Currently, BMPs are the only osteoinductive alternatives to bone grafting in the treatment of skeletal conditions necessitating surgically induced new bone formation. They are complex molecules and potent stimulators of new bone formation. However, BMPs are difficult and expensive to produce, mainly by recombinant techniques. Furthermore, they have been associated with some serious complications and side effects in clinical practice largely due to the supraphysiological doses necessary to initiate new bone formation. Due to the awareness of the potential complications and increased costs associated with the current BMP usage, there has been a large decrease in the use of BMPs by surgeons, such as the use of rhBMP-2 for the treatment of degenerative spondylolisthesis.

In addition to the stimulation of osteoblast formation, new bone formation relies on an appropriate action of bone resorbing osteoclasts. During bone grafting, a catabolic hyperactivity might lead to graft resorption before new bone has replaced the graft. Similarly, BMP therapy is associated with hone resorption effects, in vivo studies and clinical studies have shown That this increased bone resorption can be balanced with local or systemic use of bisphosphonates, resulting in a higher bone volume after bone remodeling is complete.

Bone tissue has the unique capacity to fully regenerate under normal conditions, like in the case of a traumatic fracture. The inflammatory response after bone injury determines the outcome of the subsequent bone healing process, as it triggers a complex interaction between infiltrating immune cells, resident cells and bone progenitor cells. It has been shown in knockout mice that both adaptive and innate immune cells can affect the bone healing process after bone fracture. A balanced inflammatory response seems to be critical for successful bone healing, since patients using anti-inflammatory medication or patients with a systemic and serious acute inflammation such as polytrauma, may have impaired fracture healing. Inflammatory processes can also lead to undesired new bone formation. Examples include some bacterial infections, periarticular ossification (PAO), diffuse idiopathic skeletal hyperostosis (DISH) and fibrodysplasia ossificans progressiva (FOP). In all these pathologies, limited new bone formation is likely caused by a local, yet poorly understood, inflammatory process. Since they are poorly understood, the underlying processes have not been harnessed as a clinical therapy for new bone formation.

More often, uncontrolled inflammatory processes are associated with osteolysis, the active resorption of bone matrix by osteoclasts. In bacterial infections for example, increased bone porosity and bone destruction can be observed. It is known that bacterial infections hamper the healing of bone fractures, or negatively affect spinal fusions, indicating that both bone anabolic and bone catabolic pathways are active during bone infection, with an overall negative effect on the bone tissue regeneration.

Considering the above, there is a need in the art for new strategies for the treatment of conditions requiring the induction of new bone formation. Furthermore there is a need in the art for a treatment that is cheaper, has less adverse effects on patients during treatment and is more effective than the current treatments available.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a composition for use in the treatment of an individual suffering a condition necessitating new bone formation, said composition comprising at least one immunomodulating agent and at least one bone formation promoting agent, wherein the composition induces a local inflammation. The bone formation enhancing component can be a bone anabolic and/or a bone anticatabolic component. Osteoinductive factors and osteoclast inhibiting factors are both considered bone anabolic components through direct (independent of osteoclasts), or indirect (dependent of osteoclasts) stimulation of osteoblast formation. Osteoclast inhibiting factors are also bone anticatabolic components through inhibition of osteoclast formation and activity. Present invention makes use of the modulation of the inflammatory response to enhance the effects of bone formation enhancing factors. As a response to the immunomodulation agent present in the composition of present invention, the local inflammation leads to the attraction and activation of immune cells and most likely to cytokine production by these immune cells. The local delivery of inflammatory stimuli leads to a tissue response that is stimulatory for new bone formation. For certain clinical applications, i.e. spinal fusion and large bone defects, bone (progenitor) cells should migrate to the site where new bone formation is required. If a therapeutic strategy can stimulate new bone formation independent of resident osteoblasts or osteoclasts localized in existing bone tissue, this may lead to more successful treatment in certain conditions. As an example, spinal fusions rely largely on ectopic bone formation.

According to present invention, an individual suffering a condition necessitating new bone formation, can be any vertebrate, such as a mammal, such as a human being.

Both the at least one immunomodulation agent and at least one bone formation enhancing agent are essential to present invention. Present invention approaches the normal bone regenerative process after bone damage, where immune cells orchestrate the interaction between bone-forming and bone-resorbing cells. Since osteoinductive bone substitutes or BMPs do not sufficiently target this regenerative process, present invention is aimed at modulation of the immune system to initiate or enhance the bone regenerative cascade. The immunomodulation agent component has no or limited ability to induce new bone formation, but enhances bone formation through a synergistic action together with a bone formation enhancing agent. This combined approach results in several biological processes that are activated: increased blood flow, production of pro-inflammatory cytokines, recruitment of osteoblast progenitor cells, modulation of osteoclast formation and activity, and the increased local production of cytokines and growth factors associated with osteogenesis and angiogenesis. During the bone healing process an important interaction exists between the components of the immune system and bone progenitor cells. A short and mild inflammation seems to be beneficiary for the bone formation process, whereas a lengthy and more severe/strong inflammation results in the degradation of bone tissue. An excessive local immune response (i.e. the scenario where immune cells are the major cell population in the tissue for a period of several weeks) is detrimental for bone matrix deposition and mineralization.

According to yet another preferred embodiment, the present invention relates to the composition, wherein the composition provides bone tissue generation at an ectopic location. Present invention provides several advantages compared to the state of the art. Large bone defects will heal faster by use of the invention, for example when the invention is applied in spinal fusions or non-healing bone defects in long bones. Furthermore, present invention results in that bone formation is not only affected by osteoconduction, conduction of bone formation from existing bone tissue, but also in part affected by osteoinduction, induction of bone formation in a non-bone environment. In other words, bone growth can also be stimulated where normally no bone is present. This is for instance necessary in conditions like spinal fusions, where new bone formation relies at least partially on ectopic bone formation.

According to yet another preferred embodiment, the present invention relates to the composition, wherein the composition provides bone tissue generation at the site of the condition necessitating new bone formation.

According to a preferred embodiment, the present invention relates to the composition, wherein the at least one immunomodulation agent is selected from the group consisting of inactivated bacteria, bacteria, attenuated bacteria, modified bacteria, bacterial antigens, bacterial cell wall extracts, cytokines, viral antigens, inactivated microorganisms, prostaglandins and pro-inflammatory lipoproteins, preferably inactivated bacteria. The microorganisms claimed here as part of the composition also involve microorganisms that have been (genetically or otherwise) modified before delivery to directly (e.g. production of bone stimulatory molecules) or indirectly (e.g. potentiate the desired immunomodulatory effect) enhance their bone-promoting activity. Microorganisms include bacteria, viruses, parasites, yeasts, moulds, archaea, protozoa, nematodes and algae. Furthermore, modification of the microorganism can be performed to increase safety of the therapy. Modification of the microorganisms can involve at least in part gene transfer methods to stimulate production of cytokines and growth factors by the inflamed tissue, and can also involve strains which are defective in toxin production. The method of microorganism inactivation can include both physical and chemical treatment methods, including but not limited to irradiation-, photodynamical-, heat-, electrochemical-, or chemical inactivation techniques. The method of microbial attenuation can involve but is not limited to serial passaging of the pathogen in a nonstandard host, in tissue culture or on selective compounds, by stable mutation or the deletion of one or more genes associated with virulence. The method of bacterial cell-wall extraction can involve but is not limited to physical disruption, cell grinding, heating, chemical extraction, enzyme treatment, or one or more combinations of these listed methods.

According to another preferred embodiment, the present invention relates to the composition, wherein the at least one bone formation promoting agent is selected from the group consisting of bone-stimulating growth factors, bone-stimulating bisphosphonates and/or bone antiresorptive factors. The delivery of immunomodulating agents allows for increasing the number of matrix-depositing bone cells, as well as modulating the cells involved in bone resorption. This interaction between these different components can therefore provide better bone growth.

According to yet another preferred embodiment, the present invention relates to the composition, wherein the bone-stimulating growth factors are selected from the group consisting of bone morphogenetic proteins (BMPs), BMP-2, BMP-1, BMP-3, BMP-4, BMP-5, BMP-6, BMP-7, BMP-8a, BMP-8b, BMP-9, BMP-10, BMP-15, transforming growth factor (TGF), fibroblast growth factor (FGF), vascular endothelial growth factor (VEGF) and insulin like growth factor (IGF), and heterodimers and/or isoforms thereof, preferably BMP-2.

According to a preferred embodiment, the present invention relates to the composition, wherein the bone anti-resorptive bisphosphonates are selected from the group consisting of alendronate, pamidronate, clodronate and zoledronate, preferably zoledronate.

According to another preferred embodiment, the present invention relates to the composition wherein the bone antiresorptive factors are selected from the group consisting of alendronate, pamidronate, clodronate, zoledronate, parathyroid hormone, osteoprotegerin, or SERMs (selective estrogen receptor modulators), preferably zoledronate. During the bone healing process there is a significant interaction between the components of the immune system and bone (precursor) cells. Animal studies show that whole inactivated bacteria, bacterial components, or inflammation-associated cytokines can not by themselves induce bone formation outside of the bone surrounding (ectopic sites). When inactivated bacteria or bacterial cell wall components are combined with BMPs, the combination effectively stimulates new bone formation. Also, it has been found that whole bacteria, bacterial components, or pro-inflammatory cytokines in combination with BMP-2 gives better results in comparison with BMP-2 alone. It can be concluded that whole bacteria, bacterial components, or pro-inflammatory cytokines have a synergistic effect on the BMPs in relation to the stimulation of new bone formation. Since this effect was observed for Gram-positive bacteria, Gram-negative bacteria and Mycobacteria, it is likely that a wide range of bacteria share this common synergistic effect with BMPs. When placed in the medullary cavity, inactivated bacteria induced both new bone formation and resorption of the existing bone tissue. This effect was observed for Gram-positive bacteria, Gram-negative bacteria and Mycobacteria, which suggests that an unbalance between osteoblasts and osteoclasts due to these bacteria resulted in undesired bone remodelling.

As a result of this synergistic action of these immunomodulatory components with BMP, the synergistic effect can be used in order to achieve an improved effectiveness, efficacy and stimulation of new bone formation. The advantage the present invention provides is that a much lower dose of BMP can be used in therapy in comparison to what is currently used for new bone formation using BMPs, eliminating the adverse effects currently observed in patients as a result of the high BMP dosage. Therefore, present invention also provides a possibility for the application of BMPs, which are not yet clinically applied to date but involved in normal bone regeneration, for example other types of BMPs. For BMP-2, 4, 6, 7, 9 are all shown to play an important role in skeletal development and bone healing after traumatic injury.

According to yet another preferred embodiment, the present invention relates to the composition, wherein the at least one immunomodulation agent is bacteria selected from the group of the genus of Staphylococcus, Escherichia, Mycobacterium, Bacillus and Haemophilus. More Experimental data supporting the immuno-modulatory effect of bacteria have been obtained with Staphylococcus aureus, Escherichia coli, Mycobacterium marinum, Bacillus cereus and Haemophilus influenzae.

According a preferred embodiment, the present invention relates to the composition, wherein the at least one immunomodulation agent is a bacterial antigen selected from the group consisting of lipoteichoic acid (LTA), lipoproteins, bacterial cell wall extracts, polysaccharide, lipopolysaccharide (LPS), glycolipids, or peptidoglycans, preferably LTA. LPS and LTA were studied in parallel for their effect on BMP-induced bone formation. They are the archetypical cell-wall components of Gram-negative and Gram-positive bacteria, respectively, and both have shown to modulate the osteogenic response in vivo. Also, bacterial cell wall extracts can be used as immunomodulating agent. Moreover, isolated bacterial cell wall components can be used as bacterial antigens, such as lipoproteins, polysaccharides, glycolipids, or peptidoglycans.

According to yet another preferred embodiment, the present invention relates to the composition, wherein the at least one immunomodulation agent is a pro-inflammatory cytokine selected from the group consisting of TNF-α, IL-17, IL-1, IL-6, IFN-γ and heterodimers and/or isoforms thereof. There are several candidate pro-inflammatory cytokines that can stimulate the observed osteogenesis. In present invention, pro-inflammatory cytokines act together with BMPs and other transforming growth factor family members to induce processes leading to repair and restoration. Probably these cytokines induce the osteogenic differentiation of progenitor cells.

According to yet another preferred embodiment, the present invention relates to the composition, wherein the at least one immunomodulation agent is TNF-α and the at least one bone formation promoting agent is BMP-2.The composition of present invention is preferably comprised of inactivated bacteria, TNF-α, LTA, BMP and a bone-stimulating bisphosphonate, more preferably comprised of inactivated bacteria, TNF-α, LTA and a bone-stimulating bisphosphonate, most preferably comprised of inactivated bacteria, TNF-α, LTA and BMP-2. The composition may also be comprised of inactivated bacteria and BMP-2 or TNF-α and BMP-2 or LTA and BMP-2. The composition may also be comprised of inactivated bacteria, BMP-2 and a bisphoshonate or TNF-α, BMP-2 and a bisphoshonate or LTA, BMP-2 and a bisphosphonate.

Our data show that pro-inflammatory factors do not induce a beneficial response on in vitro or in vivo osteogenesis. In contrary, our data shows that pro-inflammatory factors potentiate the action on osteogenic differentiation or bone formation. The induction of an inflammatory response of the body initiates a number of biological responses contributing to bone formation which is not observed when using bone-related growth factors alone: hyper-vascularization, trafficking of white blood cells, induction of different pro-osteogenic pathways in bone progenitor cells. Thus, pro-inflammatory stimuli by themselves do not induce new bone formation, but modify the degree of (e.g. BMP-2) induced bone formation.

According to another preferred embodiment, the present invention relates to the composition, wherein the at least one bone formation promoting agent is BMP at a concentration of between 0.1 μg/ml and 5 mg/ml, preferably of between 1 μg/ml and 1 mg/ml, most preferably of between 20 μg/ml and 500 μg/ml.

According to yet another preferred embodiment, the present invention relates to the composition, wherein the at least one immunomodulation agent is bacteria at a concentration of between 10² to 10¹² bacteria/ml, preferably 10³ to 10⁹ bacteria/ml, more preferably 10⁴ to 10⁸ bacteria/ml. Osteogenic effects due to the use of bacteria are dependent on the local concentration of proinflammatory mediators. Bacterial infection correlates strongly with the induction of new bone formation. This can result in a substantial net increase in the bone volume. No change in the bone quantity was observed in the animals that received an implant without bacterial inoculation. Stimulating bone regeneration by a controlled immune response could benefit from multiple, sequential responses, which are triggered: rapid infiltration of immune cells, local migration and differentiation of MSCs, and angiogenesis.

According to yet another preferred embodiment, the present invention relates to the composition, wherein the at least one immunomodulation agent is a bacterial antigen at a concentration of between 0.05 to 5 μg/ml. Preferably LTA is used as bacterial antigen in the composition of present invention. Increase in LTA concentration resulted in a significant increase in BMP-2 induced bone.

According to yet another preferred embodiment, the present invention relates to the composition, wherein the at least one immunomodulation agent is a cytokine at a concentration of between 0.1 to 1000 ng/ml. In present invention the cytokine is preferably a cytokine that is associated with inflammation, preferably TNF-α, IL-17, IL-1, IL-6 or IFN-γ. TNF-α is preferably used at a concentration of 0.1-100 ng/ml. IL-17 is preferably selected from its close relatives IL-17A or IL-17F, or fusion proteins comprising a part or the entire sequence of IL-17 isoform or a heterodimer of these two forms and IL-17 is preferably used at a concentration of 0.5-500 ng/ml. IL-1 is preferably IL-1β and used at a concentration of 0.1-100 ng/ml. IL-6 is preferably used at a concentration of 1-1000 ng/ml. IFN-γ is preferably used at a concentration of 0.1-100 ng/ml.

The concentration of the bone formation agent (e.g. BMP-2) and the concentration of the immunomodulation agent (e.g. inactivated bacteria, TNF-α, LTA) in the composition are selected as such that it is sufficient to obtain the desired effect. The concentrations of the bone formation agent and the concentration of the immunomodulation agent that may be used in the composition of present inventions depend on the type of bone defect (e.g. larger versus smaller bone defects) or type of applications of the composition of present invention (e.g. using a carrier that comprises the composition or directly injecting the composition at the site of the bone defect). For instance depending on the size and volume of the bone defect or when using a carrier that contains the composition of present invention, the appropriate amounts are selected by the skilled person to obtain the desired effect for the treatment of the bone defect and stimulate osteogenesis. Therefore, the concentration may also be used as amount of agent per cc carrier or cc defect volume (for example 10²-10¹² bacteria/cc carrier or 0.1 μg-5 mg BMP/cc defect volume).

According to yet another preferred embodiment, the present invention relates to the composition, wherein the condition necessitating new bone formation is selected from the group consisting of large bone defects, segmental bone defects, structural bone defects, bone fractures, non-unions of bone fractures, fusion of joints including spinal fusions and local or systemic osteoporosis. Present invention relates to compositions for the repair or regeneration of damaged bones, filling of bone defects after trauma or pathological bone loss created during surgery, and for the fusion of joints to prevent movement, including, but is not limited to orthopedics, osteolysis due to metastasis, spinal surgery, dentistry, maxillofacial surgery, traumatology, and neurosurgery.

The present invention, according to a second aspect, relates to a medicament comprising a composition of present invention.

According a preferred embodiment, the present invention relates to the medicament comprising a composition of present invention, wherein the composition comprising at least one immunomodulation agent and at least one bone formation promoting agent, wherein the composition induces a local inflammation.

According to another preferred embodiment, the present invention relates to the medicament comprising a composition of present invention, wherein the composition provides bone tissue generation at an ectopic location. The ectopic location may be a non-bone location or partially ectopic location. For instance, a spinal fusion is regarded as a partially ectopic location.

According to yet another preferred embodiment, the present invention relates to the medicament comprising a composition of present invention, wherein the composition provides bone tissue generation at the site of the condition necessitating new bone formation.

According to yet another preferred embodiment, the present invention relates to the medicament comprising a composition of present invention, wherein the at least one immunomodulation agent is selected from the group consisting of inactivated bacteria, bacteria, attenuated bacteria, modified bacteria, bacterial antigens, bacterial cell wall extracts, cytokines, viral antigens, inactivated microorganisms, prostaglandins and pro-inflammatory lipoproteins, preferably inactivated bacteria.

According to another preferred embodiment, the present invention relates to the medicament comprising a composition of present invention, wherein the at least one bone formation promoting agent is selected from the group consisting of bone-stimulating growth factors, bone-stimulating bisphosphonates and bone antiresorptive factors.

According to a preferred embodiment, the present invention relates to the medicament comprising a composition of present invention, wherein the bone-stimulating growth factors are selected from the group consisting of bone morphogenetic proteins (BMPs), BMP-2, BMP-1, BMP-3, BMP-4, BMP-5, BMP-6, BMP-7, BMP-8a, BMP-8b, BMP-9, BMP-10, BMP-15, transforming growth factor (TGF), fibroblast growth factor (FGF), vascular endothelial growth factor (VEGF), insulin like growth factor (IGF), and fusion proteins, heterodimers and/or isoforms thereof, preferably BMP-2.

According to yet another preferred embodiment, the present invention relates to the medicament comprising a composition of present invention, wherein the bone-stimulating bisphosphonates are selected from the group consisting of alendronate, pamidronate, clodronate and zoledronate, preferably zoledronate.

According to another preferred embodiment, the present invention relates to the medicament comprising a composition of present invention, wherein the bone antiresorptive factors are selected from the group consisting of alendronate, pamidronate, clodronate, zoledronate, parathyroid hormone, osteoprotegerin, or SERMs (selective estrogen receptor modulators), preferably zoledronate.

According to another preferred embodiment, the present invention relates to the medicament comprising a composition of present invention, wherein the at least one immunomodulation agent is bacteria selected from the group of the genus of Staphylococcus, Escherichia, Mycobacterium, Bacillus and Haemophilus.

According to a preferred embodiment, the present invention relates to the medicament comprising a composition of present invention, wherein the at least one immunomodulation agent is a bacterial antigen selected from the group consisting of lipoteichoic acid (LTA), lipoproteins, bacterial cell wall extracts, polysaccharide, lipopolysaccharide (LPS), glycolipids, or peptidoglycans, preferably LTA.

According to another preferred embodiment, the present invention relates to the medicament comprising a composition of present invention, wherein the at least one immunomodulation agent is a pro-inflammatory cytokine selected from the group consisting of TNF-α, IL-17, IL-1, IL-6, IFN-γ, and heterodimers and/or isoforms thereof.

According to yet another preferred embodiment, the present invention relates to the composition, wherein the at least one immunomodulation agent is TNF-α and the at least one bone formation promoting agent is BMP-2.

According to yet another preferred embodiment, the present invention relates to the medicament comprising a composition of present invention, wherein the at least one bone formation promoting agent is BMP at a concentration of between 0.1 μg/ml and 5 mg/ml, preferably of between 1 μg/ml and 1 mg/ml, most preferably of between 20 μg/ml and 500 μg/ml.

According to yet another preferred embodiment, the present invention relates to the medicament comprising a composition of present invention, wherein the at least one immunomodulation agent is bacteria at a concentration of between 10² to 10¹² bacteria/ml, preferably 10³ to 10⁹ bacteria/ml, more preferably 10⁴ to 10⁸ bacteria/ml.

According to a preferred embodiment, the present invention relates to the medicament comprising a composition of present invention, wherein the at least one immunomodulation agent is bacterial antigen at a concentration of between 0.05 to 5 μg/ml.

According to another preferred embodiment, the present invention relates to the medicament comprising a composition of present invention, wherein the at least one immunomodulation agent is a cytokine at a concentration of between 0.1 to 1000 ng/ml.

According to yet another preferred embodiment, the present invention relates to the medicament comprising a composition of present invention, wherein the condition necessitating new bone formation is selected from the group consisting of large bone defects, segmental bone defects, structural bone defects, bone fractures, non-unions of bone fractures, fusion of joints including spinal fusions, and osteoporosis.

The present invention, according to a third aspect, relates to an osteoconductive carrier provided with a composition of present invention, wherein the carrier is comprised of collagen, natural or synthetic hydrogel or polymer, calcium hydroxyapatite, calcium phosphate, calcium silicates, calcium sulphates, bioglass, titanium, tantalum and/or stainless steel. The carrier can be produced using various production techniques known in the art, including 3D-printing of the carrier. Carriers, such as biomimetic scaffolds are synthetic products, usually of a metal or a ceramic, which are used for stimulation of bone growth. The carriers do not initiate new bone formation (osteoinduction), but are usually osteoconductive, facilitating only the growth of bone into or through the scaffolds. They are not as effective as the use of autologous bone or BMPs. The compositions are delivered locally to the patient, with or without a carrier material with the intention of promoting new bone formation through a local inflammation process. The composition of present invention can be used in combination with these scaffolds to create osteoinductive materials that can initiate new bone formation. As such, the scaffolds provide structural support and a surface that guides new bone formation. The composition can be constituted into a gel, paste, solvent, putty-type or other type of consistency.

According a preferred embodiment, the present invention relates to the osteoconductive carrier provided with a composition of present invention, wherein the composition comprising at least one immunomodulation agent and at least one bone formation promoting agent, wherein the composition induces a local inflammation.

According to another preferred embodiment, the present invention relates to the osteoconductive carrier provided with a composition of present invention, wherein the composition provides bone tissue generation at an ectopic location.

According to yet another preferred embodiment, the present invention relates to the osteoconductive carrier provided with a composition of present invention, wherein the composition provides bone tissue generation at the site of the condition necessitating new bone formation.

According to yet another preferred embodiment, the present invention relates to the osteoconductive carrier provided with a composition of present invention, wherein the at least one immunomodulation agent is selected from the group consisting of inactivated bacteria, bacteria, attenuated bacteria, modified bacteria, bacterial antigens, bacterial cell wall extracts, cytokines, viral antigens, inactivated microorganisms, prostaglandins and pro-inflammatory lipoproteins, preferably inactivated bacteria.

According to another preferred embodiment, the present invention relates to the osteoconductive carrier provided with a composition of present invention, wherein the at least one bone formation promoting agent is selected from the group consisting of bone-stimulating growth factors, bone-stimulating bisphosphonates and bone antiresorptive factors.

According to a preferred embodiment, the present invention relates to the osteoconductive carrier provided with a composition of present invention, wherein the bone-stimulating growth factors are selected from the group consisting of bone morphogenetic proteins (BMPs), BMP-2, BMP-1, BMP-3, BMP-4, BMP-5, BMP-6, BMP-7, BMP-8a, BMP-8b, BMP-9, BMP-10, BMP-15, transforming growth factor (TGF), fibroblast growth factor (FGF), vascular endothelial growth factor (VEGF), insulin like growth factor (IGF), and fusion proteins, heterodimers and/or isoforms thereof, preferably BMP-2.

According to yet another preferred embodiment, the present invention relates to the osteoconductive carrier provided with a composition of present invention, wherein the bone-stimulating bisphosphonates are selected from the group consisting of alendronate, pamidronate, clodronate and zoledronate, preferably zoledronate.

According to another preferred embodiment, the present invention relates to the osteoconductive carrier provided with a composition of present invention, wherein the bone antiresorptive factors are selected from the group consisting of alendronate, pamidronate, clodronate, zoledronate, parathyroid hormone, osteoprotegerin, or SERMs (selective estrogen receptor modulators), preferably zoledronate.

According to another preferred embodiment, the present invention relates to the osteoconductive carrier provided with a composition of present invention, wherein the at least one immunomodulation agent is bacteria selected from the group of the genus of Staphylococcus, Escherichia, Mycobacterium, Bacillus and Haemophilus.

According to a preferred embodiment, the present invention relates to the osteoconductive carrier provided with a composition of present invention, wherein the at least one immunomodulation agent is a bacterial antigen selected from the group consisting of lipoteichoic acid (LTA), lipoproteins, bacterial cell wall extracts, polysaccharide, lipopolysaccharide (LPS), glycolipids, or peptidoglycans, preferably LTA.

According to another preferred embodiment, the present invention relates to the osteoconductive carrier provided with a composition of present invention, wherein the at least one immunomodulation agent is a pro-inflammatory cytokine selected from the group consisting of TNF-α, IL-17, IL-1, IL-6, IFN-γ and fusion protein, heterodimers and/or isoforms thereof. According to yet another preferred embodiment, the present invention relates to the composition, wherein the at least one immunomodulation agent is TNF-α and the at least one bone formation promoting agent is BMP-2.

According to yet another preferred embodiment, the present invention relates to the osteoconductive carrier provided with a composition of present invention, wherein the at least one bone formation promoting agent is BMP at a concentration of between 0.1 μg/ml and 5 mg/ml, preferably of between 1 μg/ml and 1 mg/ml, most preferably of between 20 μg/ml and 500 μg/ml.

According to yet another preferred embodiment, the present invention relates to the osteoconductive carrier provided with a composition of present invention, wherein the at least one immunomodulation agent is bacteria at a concentration of between 10² to 10¹² bacteria/ml, preferably 10³ to 10⁹bacteria/ml, more preferably 10⁴ to 10⁸ bacteria/ml.

According to a preferred embodiment, the present invention relates to the osteoconductive carrier provided with a composition of present invention, wherein the at least one immunomodulation agent is bacterial antigen at a concentration of between 0.05 to 5 μg/ml.

According to another preferred embodiment, the present invention relates to the osteoconductive carrier provided with a composition of present invention, wherein the at least one immunomodulation agent is a cytokine at a concentration of between 0.1 to 1000 ng/ml.

According to yet another preferred embodiment, the present invention relates to the osteoconductive carrier provided with a composition of present invention, wherein the condition necessitating new bone formation is selected from the group consisting of large bone defects, segmental bone defects, structural bone defects, bone fractures, non-unions of bone fractures, fusion of joints including spinal fusions, and osteoporosis.

The present invention, according to a fourth aspect, relates to methods for the treatment of an individual suffering from a condition necessitating new bone formation, comprising administering to said individual a composition of present invention.

According to a preferred embodiment, the present invention relates to methods wherein the composition of present invention is administered via a local injection or via a carrier as defined in the appended claims that is implanted in said individual. Depending on the application, the compositions described herein can be administered to the patient in different forms. These include, but are not limited to, injection of solutions and gels or surgical implantation of biomaterial carriers loaded with the composition. Carrier materials can be calcium phosphates, metals or natural bone products.

The present invention, according to a further aspect, relates to the use of the composition of present invention for the coating of orthopaedic instruments and materials. Materials used during surgery, such as nails, pins, rods, screws and plates used to anchor fractured bones while they heal or to replace a missing joint or bone or to support a damaged bone may be coated with the composition of present invention.

The present invention will be further detailed in the following examples, in the examples reference is made to figures wherein:

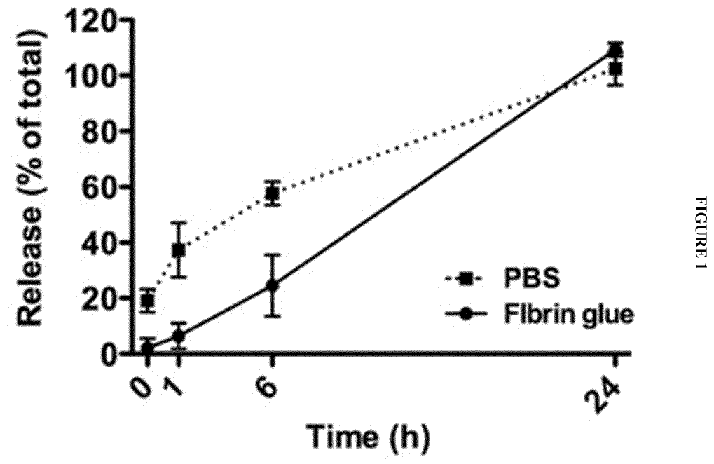

FIG. 1: shows the in vitro release of LPS from BCP constructs. This LPS was loaded in fibrin glue or in PBS, and its concentration was measured in the supernatant at different time points;

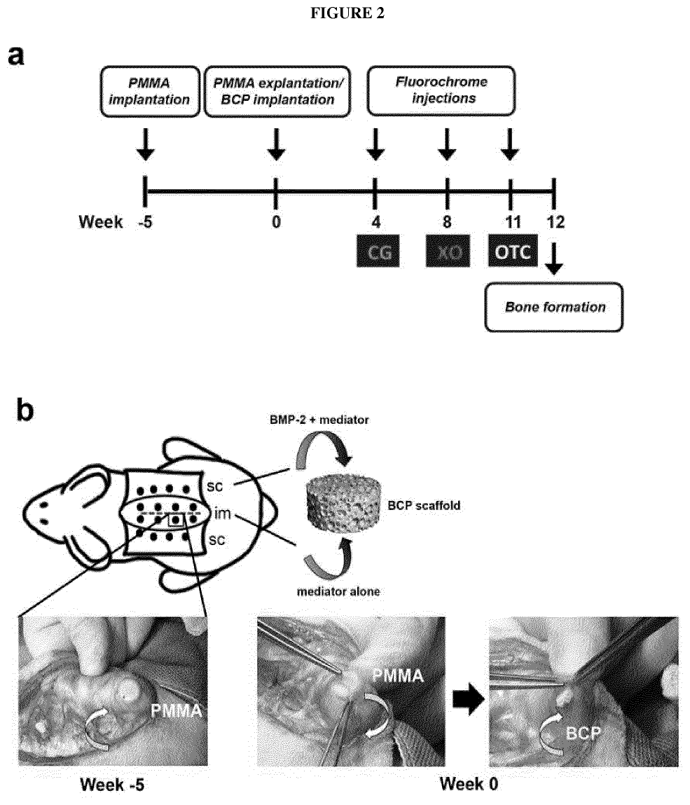

FIG. 2: shows the design of the comparative study to investigate local bone formation in conjunction with inflammatory stimuli (a). Timeline showing the relative time points for the different interventions. Bone formation was the primary outcome after 12 weeks. Schematic representation of the subcutaneous (sc) en intramuscular (im) implantations in the rabbits (b). The mediators were loaded onto biphasic calcium phosphate (BCP) scaffolds and studied alone, or in combination with bone morphogenetic protein (BMP)-2. These constructs were implanted into 5-week old poly methyl methacrylate (PMMA)-induced biomembrane pockets;

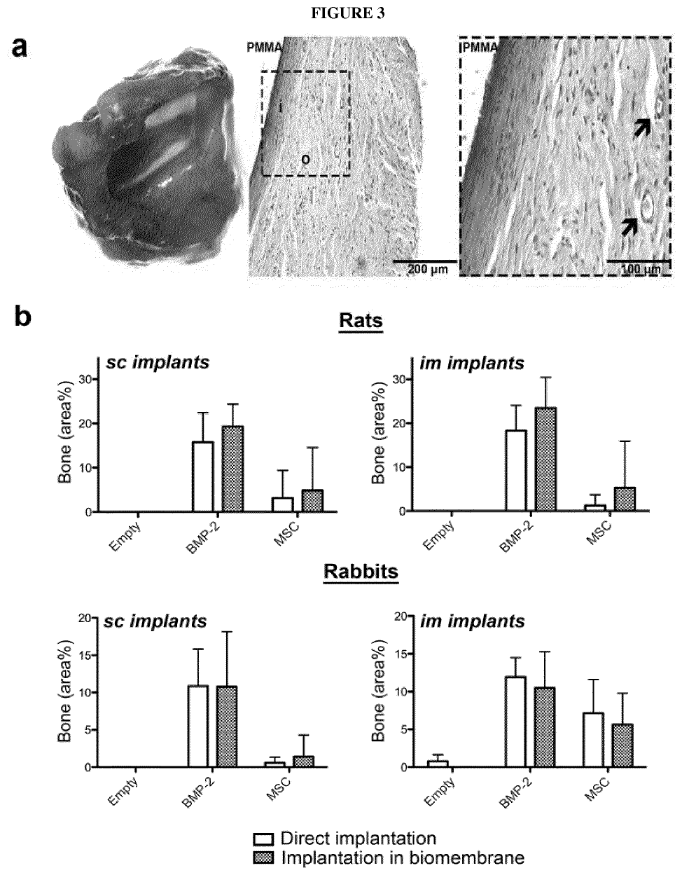

FIG. 3: shows the results of the assessment study to evaluate the ectopic biomembrane pocket model. Macroscopic and histological appearance of the poly methyl methacrylate (PMMA)-induced biomembrane pockets created subcutaneously in rabbits (a). H&E staining shows the formation of a thick outer membrane (0) of connective tissue with an endothelium-like inner lining (i) after 6 weeks. Blood vessels were observed in the outer membrane (arrows). Bone formation in the constructs following direct implantation in fresh pockets, or implantation in the pre-induced biomembrane pockets (b). The pockets were created both subcutaneously and intramuscularly in rats and rabbits (n=4). Constructs containing BMP-2 (3.75 μg in rats, 10.5 μg in rabbits) or MSCs (2.5×10⁵ in rats, 7×10⁵ cells in rabbits) were compared to empty constructs. The results are represented as the mean±standard deviation;

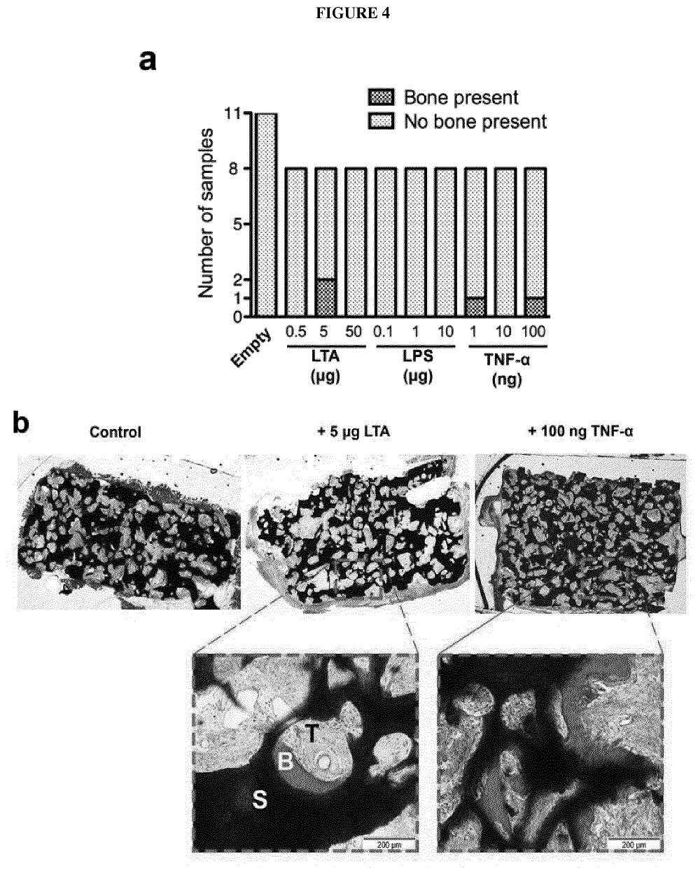

FIG. 4: shows the bone induction in intramuscularly implanted constructs in rabbits. Scoring for the presence of bone tissue in the different groups (a). Histology of sections stained with methylene blue and basic fuchsin reveals only limited spots of bone (bright pink) (b). B: bone, S: scaffold material. T: fibrous tissue ingrowth;

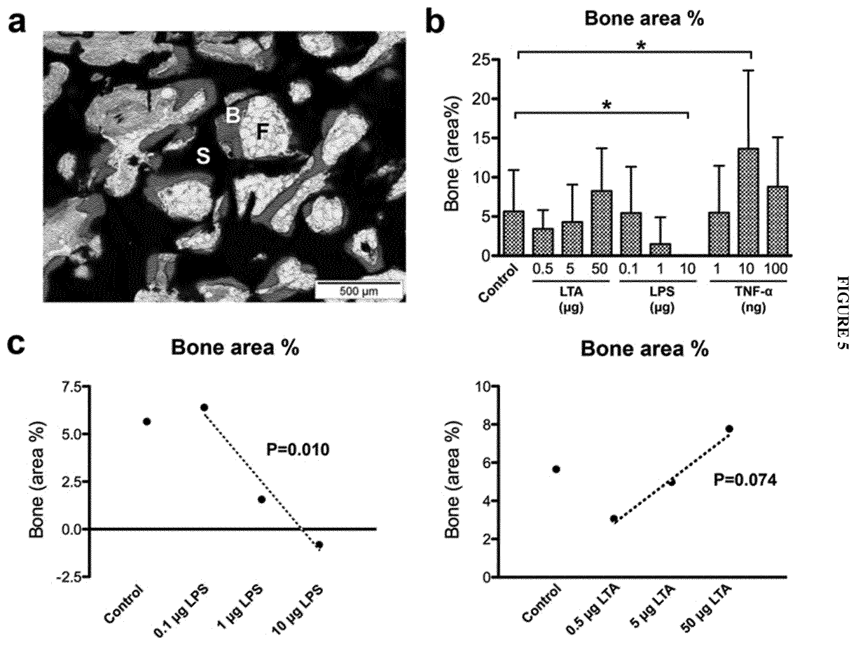

FIG. 5: shows the bone formation in subcutaneously implanted samples in rabbits. Histology of methylene blue and basic fuchsin-stained sections, representative for the control samples with 1.5 μg BMP-2 alone (a). Bone tissue is stained bright pink. B: bone, S: scaffolds material. F: fat tissue. Quantification of the amount of bone in the constructs (b). The results are represented as the mean±standard deviation. *p<0.05 compared to the control with 1.5 μg BMP-2 alone. Dose-dependent effects of LPS and LTA on new bone formation (c). Regression analyses show the linear relationship between the mediator concentration and the amount of bone in subcutaneously implanted constructs. The estimated means are shown together with the trend line;

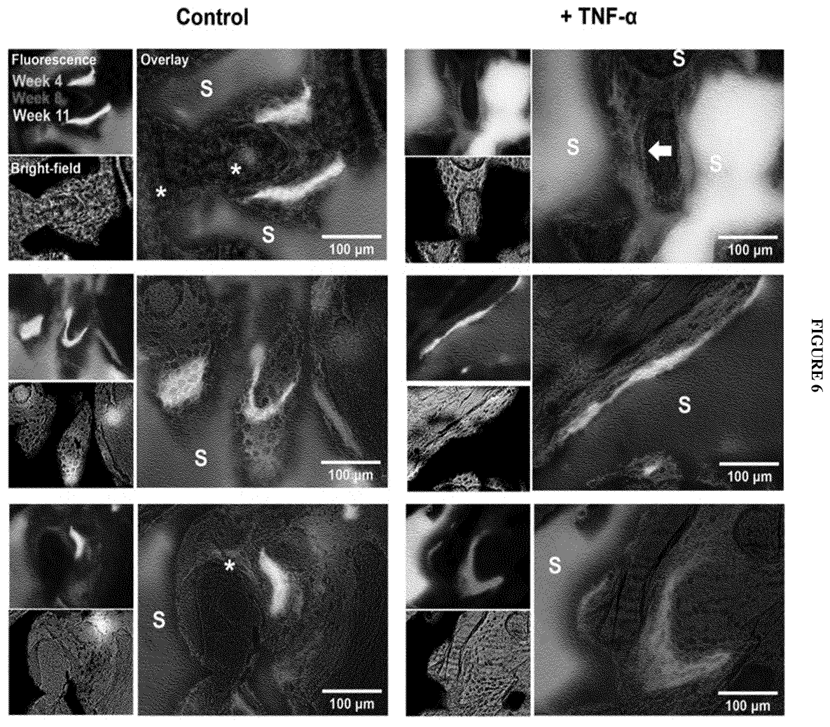

FIG. 6: shows the fluorochrome incorporation in subcutaneously implanted samples in rabbits. Fluorochromes were injected at 4 (calcein, green), 8 (xylenol orange, red), and 11 (oxytetracycline, yellow) weeks. Their incorporation in new bone was assessed by fluorescence microscopy. Left panel: 1.5 μg BMP-2 alone. Right panel: 1.5 μg BMP-2 and 10 ng TNF-α. The upper, middle and lower panels are representative for three rabbits. Oxytetracycline incorporation (arrow) was only occasionally seen as a thin line, not to be confused with the background as a result of image merging (asterisks). S: scaffold;

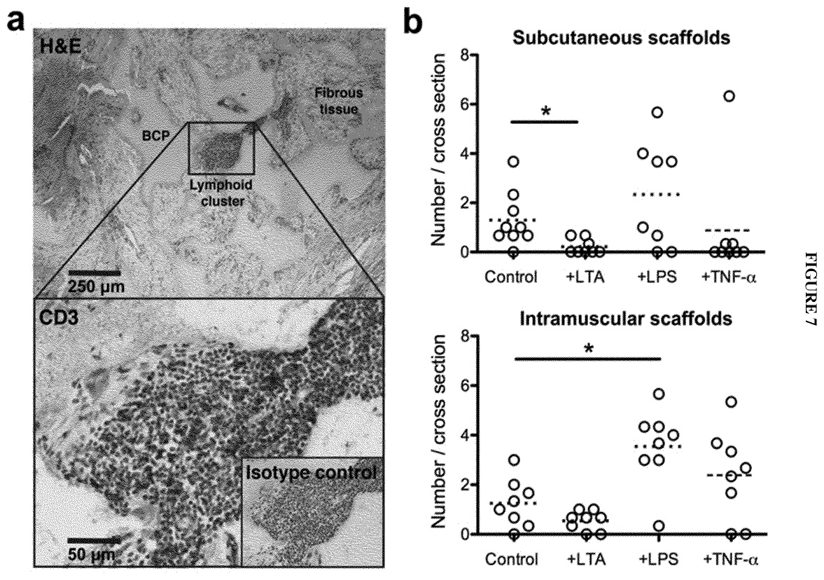

FIG. 7: shows the presence of lymphoid cell clusters in the constructs after 12 weeks in rabbits. Staining for CD3 shows the presence of T cells within the lymphoid clusters (inset stained with isotype matched control antibody) (a). Lymphoid cluster scores for each experimental group together with the group mean (b). *p<0.05 compared to the control group;

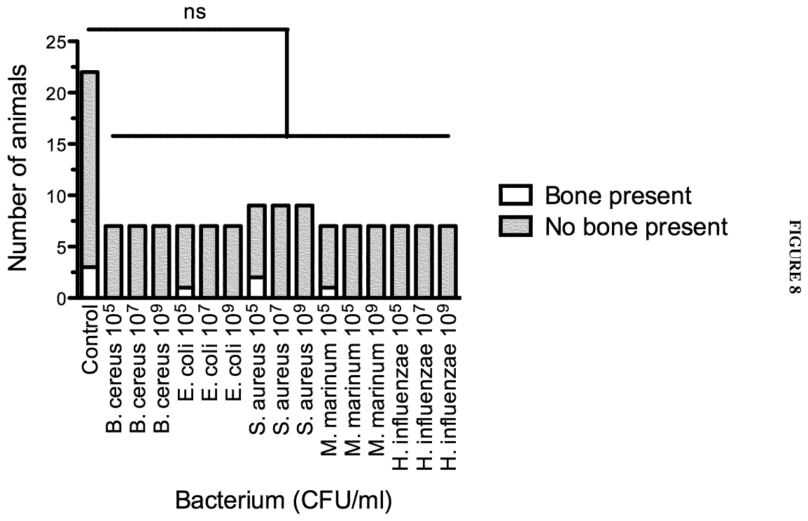

FIG. 8: shows that inactivated bacteria do not induce ectopic bone formation. Porous ceramic scaffolds were loaded with different concentrations of whole inactivated bacteria [S. aureus (10⁵-10⁹ CFU/ml, n=9), B. cereus (10⁵-10⁹ CFU/ml, n=7), E. coli (10⁵-10⁹ CFU/ml, n=7), H. influenzae (n=7), M. marinum (n=7)]. As a control, the scaffolds were loaded with PBS. The scaffolds were implanted in intramuscular pockets in the rabbits. Bone formation was assessed after 8 weeks;

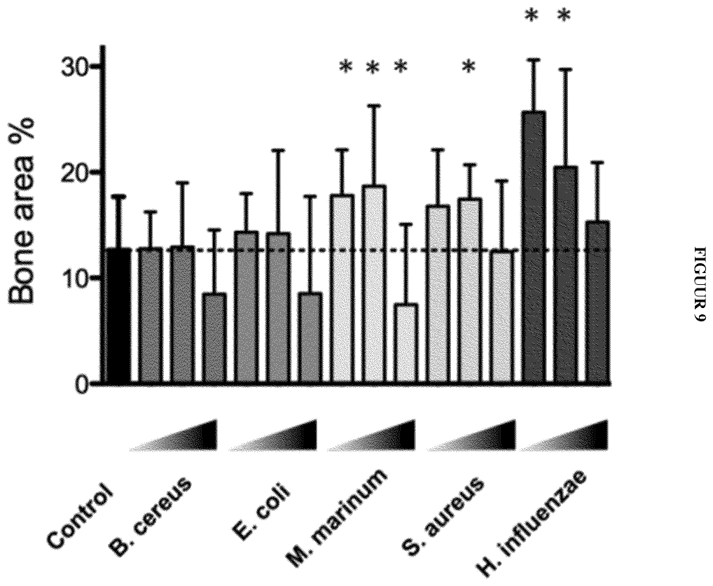

FIG. 9: shows the bone formation after 8 weeks in biphasic calcium phosphate scaffolds implanted subcutaneously in rabbits. Control: 21.5 μg/ml BMP-2. Other groups: 21.5 μg/ml BMP-2+gamma-inactivated bacteria in three concentrations. *p<0.05 compared to control.

### METHODS (FIGS. 1-7, TABLES 1 & 2)

### Study Design

First, an assessment study was performed to verify the potential for ectopic bone formation in the biomembrane pocket model. Established constructs for ectopic bone formation, i.e. porous BCP scaffolds loaded with MSCs or BMP-2, were implanted intramuscularly and subcutaneously in rats (n=4) and rabbits (n=4), inside and outside the biomembrane pockets. The bone volume (area%) in the BCP constructs was the primary outcome after 8 (rats) or 12 (rabbits) weeks. Subsequently, the rabbit model (n=11) was used to 1) screen for the potential of inflammatory stimuli to induce bone intramuscularly and to 2) screen for the effects of inflammatory stimuli on BMP-induced bone formation subcutaneously. The bone volume (area%) in the BCP constructs was the primary outcome after 12 weeks.

### Materials

Poly methyl methacrylate (PMMA, Simplex P, Stryker, Kalamazoo, USA) discs were produced using custom-made silicone molds. The PMMA discs were sterilised in a 1 M NaOH solution and extensively washed with PBS. Biphasic calcium phosphate (BCP) blocks were made with dimensions of 3.5×3.5×3 mm (rats) or 6×6×3 mm (rabbits). These BCP blocks consisted of 20±5% β-tricalcium phosphate and 80±5% hydroxyapatite by weight, and had a total porosity of 75±5% (Yuan et al., 2002). The BCP blocks were autoclaved at 121° C. for 30 min and dried at 60° C.

The following inflammatory mediators were tested for their effect on bone formation: human tumor necrosis factor alpha (TNF-α, 14-8329, Ebioscience, San Diego, USA), Lipopolysaccharide (LPS, E. coli, L5418, Sigma, St. Louis, USA), and Lipoteichoic Acid (LTA, S. aureus, L2515, Sigma). These mediators were tested with or without recombinant human BMP-2 (InductOS, Wyeth/Pfizer, New York, USA).

### Mesenchymal Stem Cell Isolation and Culture

For syngeneic rat MSC transplantations, one rat was killed using an overdose of CO₂. Using a sterile technique, the bone marrow was flushed from the femurs. For autologous rabbit MSC transplantation, bone marrow was harvested from each animal under general anesthesia by aspiration from the iliac crest using an 18G needle. The mononuclear cell fraction was isolated by Ficoll-paque centrifugation and plated in expansion medium, consisting of α-MEM (Invitrogen, Carlsbad, Calif., USA) supplemented with 10% (v/v) heat-inactivated fetal bovine serum (Cambrex, East Rutherford, N.J., USA), 0.2 mM L-ascorbic acid 2-phosphate (Sigma), and 100 units/ml penicillin/100 μg/ml streptomycin (Invitrogen). The cell cultures were kept in a humidified incubator at 37° C. and 5% CO₂. The adherent cells were expanded to passage 4 and cryopreserved. The cells were replated and cultured for two days before in vivo implantation.

### Construct Preparation

For the assessment study (Table 1), BCP scaffolds were loaded with MSCs (10⁷ cells/mi), BMP-2 (150 μg/ml) or PBS (empty control) in a volume of 25 μl for rats, or in a volume of 70 μl for rabbits. After two hours in a humidified incubator, the constructs were submerged in medium and stored overnight at 37° C. and 5% CO2.

TABLE 1
Conditions implanted both in pre-induced biomembrane pockets
and in fresh pockets (assessment study in rats and rabbits).

| | MSCs/BMP-2 | | | | |
|---|---|---|---|---|---|
| Group | (Dose) | (Conc) | n | PMMA size | BCP size |

Rat subcutaneous and intramuscular groups
(6 week PMMA + 8 week BCP)

| | | | | | |
|---|---|---|---|---|---|
| Empty control | — | — | 4 | 6 mm Ø × 4 mm | 3.5 × 3.5 × 3 mm |
| Syngeneic MSCs | 2.5 × 10⁵ | 10⁷/ml | 4 | | |
| BMP-2 | 3.75 μg | 150 μg/ml | 4 | | |

Rabbit subcutaneous and intramuscular groups
(6 week PMMA + 12 week BCP)

| | | | | | |
|---|---|---|---|---|---|
| Empty control | — | — | 4 | 10 mm Ø × 4 mm | 6 × 6 × 3 mm |
| Autologous MSCs | 7 × 10⁵ | 10⁷/ml | 4 | | |
| BMP-2 | 10.5 μg | 150 μg/ml | 4 | | |

BMP-2: bone morphogenetic protein 2, BCP: biphasic calcium phosphate, MSC: mesenchymal stem cell, PMMA: poly methyl methacrylate.

For the comparative study (Table 2), inflammatory stimuli were added to the BCP, alone or in combination with BMP-2. Either 1.5 μg of BMP-2 or PBS (15 μl volume) was first applied. Fibrin glue (Tissucol 500®, Baxter, Deerfield, Ill., USA) was subsequently used (55 μl volume) to load the inflammatory mediators onto the constructs. For this purpose, the mediators were resuspended in 27.5 μl of the thrombin component (diluted 1:50 in PBS), then mixed with 27.5 μl of the fibrinogen component (diluted 1:30 in PBS) and immediately pipetted onto the BCP scaffolds. The control samples were treated with fibrin glue alone. The constructs were prepared on the day of surgery and stored in a humidified environment at 37° C. until implantation. To determine the release profile, BCP scaffolds were loaded with LPS in fibrin glue. As a control, LPS was loaded onto the scaffolds in PBS. The constructs were kept in PBS at 37° C. At different time points, samples were taken from the PBS to determine the LPS concentration using a LAL endotoxin assay according to the manufacturer's protocol (Genscript, Piscataway, N.J., USA). It was found that the LPS was better retained using the fibrin glue method during the first hours, although the LPS was completely released from the constructs within 24 hours in both conditions (FIG. 1).

TABLE 2
Conditions implanted in pre-induced biomembrane
pockets (comparative study in rabbits).

| | BMP-2 | | Mediator | | |
|---|---|---|---|---|---|
| Group | (Dose) | (Conc) | (Dose) | (Conc) | n |

Rabbit intramuscular groups
(5 week PMMA + 12 week BCP)

| Control | x | x | | 11 |
|---|---|---|---|---|
| TNF-α | x | 1, 10, 100 ng | 14.3, 143, 1430 ng/ml | 8 |
| LPS | x | 0.1, 1, 10 μg | 1.4, 14.3, 143 μg/ml | 8 |
| LTA | x | 0.5, 5, 50 μg | 7.1, 71, 710 μg/ml | 8 |

Rabbit subcutaneous groups
(5 week PMMA + 12 week BCP)

| Control | 1.5 μg | 22 μg/ml | x | | 11 |
|---|---|---|---|---|---|
| TNF-α | 1.5 μg | 22 μg/ml | 1, 10, 100 ng | 14.3, 143, 1430 ng/ml | 8 |
| LPS | 1.5 μg | 22 μg/ml | 0.1, 1, 10 μg | 1.4, 14.3, 143 μg/ml | 8 |
| LTA | 1.5 μg | 22 μg/ml | 0.5, 5, 50 μg | 7.1, 71, 710 μg/ml | 8 |

BMP-2: bone morphogenetic protein 2, BCP: biphasic calcium phosphate, TNF-α: tumor necrosis factor alpha, LPS: lipopolysaccharide, LTA: lipoteichoic acid.

### Animals

Animal experiments were performed after approval of the local Ethics Committee for Animal Experimentation and in compliance with the Institutional Guidelines on the use of laboratory animals (Utrecht University, Utrecht, The Netherlands). A total of fifteen male New Zealand white rabbits (14 weeks old, 2.5-3.0 kg, Crl:KBL, Charles River, L'Arbresle, France) and five male Fischer rats (14 weeks old, 300-350 g, F344/IcoCrl, Charles River) were used for the experiments. Four rabbits and five rats were used to evaluate the ectopic bone formation within the induced biomembrane (assessment study). Eleven rabbits were used to study the effect of inflammatory stimuli on ectopic bone induction and formation (comparative study). All animals were housed at the Central Laboratory Animal Institute, Utrecht University. They were allowed to acclimatise for at least two weeks before the surgery. Food and water were given ad libitum.

### Surgical Procedure

The animals underwent two surgeries under general anesthesia as part of the two-step biomembrane pocket model (FIG. 2). Rats received 3% isoflurane, while rabbits received Ketamine (15 mg/kg i.m.; Narketan®, Vétoquinol BV, 's-Hertogenbosch, The Netherlands) and Glycopyrrolate (0.1 mg/kg i.m.; Robinul, Riemser Arzneimittel AG, Greifswald, Germany) preoperatively, and Medetomidine (0.25 mg/kg s.c.; Dexdomitor®, Orion Corporation, Espoo, Finland) perioperatively. Anesthesia was reversed with Atipamezole hydrochloride (0.5-1.0 mg/kg i.v., Atipam® (Eurovet Animal Health B.V., Bladel, The Netherlands). The rabbits received antibiotic prophylaxis with Enroflaxicine (10 mg/kg sc; Baytril®, Baytril, Leverkusen, Germany) once daily for three days perioperative during the first surgery, and Penicillin (3×104 IE benzylpenicilline/kg, Duplocilline®, Merck Animal Health, Madison, USA) once during the second surgery Animals were given relief preoperatively, and postoperatively every 8 hours for 2 days with Buprenorphine (0.03 mg/kg s.c.; Temgesic®, RB Pharmaceuticals Limited, Slough, UK).

After shaving and disinfecting the skin with 10% povidone-iodine, a midline incision was made to expose the paraspinal muscles. In the rabbits, four intramuscular pockets were created on each side by blunt dissection for implantation of the PMMA discs (FIG. 2B). The same discs were implanted in subcutaneous pockets via the same approach. Pockets were closed with a non-resorbable suture (Prolene®, Ethicon, USA), followed by closure of the skin (Monocryl®, Ethicon). At the second surgery, an incision was made in the membrane surrounding the PMMA disc, the disc was replaced by a BCP construct, and the opening was sutured (Prolene®, Ethicon). For the assessment study, the same constructs were simultaneously implanted in freshly prepared intramuscular and subcutaneous pockets during the second surgery. Furthermore, as part of the assessment study, two subcutaneous biomembranes were explanted from each animal during the second surgery and fixated in formalin for histology. Fluorochrome labels were injected subcutaneously to determine the onset and location of new bone formation (van Gaalen et al., 2010): calcein green (10 mg/kg s.c. in 0.2 M NaHCO3, Sigma), oxytetracycline (25 mg/kg s.c. in 50/50 PBS/demineralised water, Merck Millipore, Billerica, USA) xylenol orange (30 mg/kg s.c. in 0.12 M NaHCO3, Sigma). The incorporation of fluorochromes in the bone was examined by fluorescence microscopy on methyl methacrylate-embedded sections. The rabbits were killed 12 weeks after the second surgery under general anesthesia, by intravenous Pentobarbital (Euthanimal®, Alfasan Nederland BV, Woerden, The Netherlands). The rats were killed with CO2, 8 weeks after the second surgery.

### Bone Histomorphometry

After retrieval of the constructs, a quarter of each sample was removed for decalcification and paraffin embedding. The remaining material was fixed in 4% paraformaldehyde, dehydrated by an ethanol series and embedded in methyl methacrylate (MMA, Merck Millipore). Subsequently, 35 μm-thick sections were cut using a sawing microtome (Leica, Nusslochh, Germany) and stained with basic fuchsin and methylene blue. The samples were completely sectioned and scored for the presence of bone. Two mid-sections were pseudo-coloured in Adobe Photoshop CS6 (Adobe Systems, San Jose, USA) to quantify the percentage of bone in the available pore space (bone area %). The mean value of two sections was used for further statistical analyses. For four rabbits, one MMA section per BCP sample was left unstained for fluorochrome detection by fluorescence microscopy (Olympus BX51 with DP70 camera, Olympus, Shinjuku, Tokyo, Japan).

### CD3 Immunohistochemical Staining

Sections of decalcified (0.3 M EDTA) BCP samples were embedded in paraffin and cut into 6 μm sections. The sections were treated with 0.1% (w/v) proteinase K for 15 min. Blocking was performed with 0.3% (v/v) H2O2 in PBS for 10 min and 5% bovine serum albumin (BSA) for 30 min. Sections were then incubated with a mouse-anti-human CD3 antibody (0.7 mg/ml, M7254, clone F7.2.38, Dako, Glostrup, Denmark) for 2 h at room temperature. A mouse IgG1 antibody (X0931, Dako) was used as an istoype control. Sections were incubated with the secondary goat-anti-mouse-HRP (5 μg/ml, P0447, Dako) for 30 min at room temperature and subsequently with 3,3′-diaminobenzidine tetrahychloride hydrate (DAB, D5637, Sigma). The number of lymphoid clusters was scored on H&E stained sections by three researchers on blinded samples. A low interobserver variation in the counts was noted, and therefore the average of the three counts was used for further analyses.

### Statistical Analyses

For the assessment study, an arbitrary sample size of 4 was chosen. All conditions could be implanted in the same animal, thus requiring 4 animals for this study. For the comparative study, a sample size calculation was performed for the bone area% as main outcome parameter. This showed that a sample size of 8 was needed, based on an estimated effect size of 30% with a standard deviation of 15% (Reikeras et al., 2005), using a power of 80% and an alpha of 5% for pairwise comparisons. Since not all conditions could be implanted in the same animal, 11 rabbits were required for this experiment. All results are shown as the mean±standard deviation. Statistics were performed using SPSS version 20.0 (IBM, Chicago, USA). Differences in bone area % were analysed using a linear mixed-model approach. One-way ANOVA was used to analyse differences in the average number of lymphoid clusters. Bonferroni correction was used for multiple comparisons. Mixed model regression analysis was used to determine dose response relationships. The significance of intramuscular bone induction was analysed with a linear mixed-model approach with binary outcome measure (i.e. bone or no bone).

### Results (FIGS. 1-7)

### Biomembrane Pocket Characteristics and Influence on Bone Formation

A clear biomembrane had formed around the implanted PMMA discs in a period of 6 weeks (FIG. 3A). The biomembrane pockets had similar characteristics in rats and rabbits. They appeared well vascularised by macroscopic evaluation, while microscopically, H&E staining showed the formation of a 300-400 μm thick membrane. The membranes consisted of a cell-rich inner connective tissue layer with a thickness of 20-50 μm. Here, the fibroblasts and collagen fibers were arranged parallel to the surface of the discs. The outer part of the biomembranes consisted of a thicker layer of loose connective tissue, containing larger blood vessels. No signs of chronic inflammation were observed by histological evaluation for lymphoid cell clusters or by staining for foreign body giant cells. The membrane had appropriate biomechanical properties for handling and suturing, and provided enough space for the implantation of the BCP scaffolds.

To assess bone formation within the biomembrane, osteogenic constructs (Table 1) were implanted within the induced biomembrane pockets, or in fresh pockets. After 8 (rats) or 12 (rabbits) weeks, the bone area % did not obviously differ for any of the conditions when comparing the biomembrane pockets to the fresh pockets (FIG. 3) Whereas empty BCP constructs failed to induce any bone formation ectopically in rats (FIG. 3B, upper panel), minute spots of bone were present in 3/4 empty BCP samples in rabbits implanted in the fresh muscle pockets (FIG. 3B, lower panel).

### Effects of Inflammatory Stimuli on Bone Induction

The intramuscularly-implanted constructs showed no signs of scaffold degradation. Scoring of the empty control scaffolds showed no bone formation after 12 weeks (FIG. 4A). In addition, none of the constructs loaded with LPS demonstrated new bone formation. Although TNF-α induced bone formation in 2 constructs, no concentration dependence was observed. LTA at the dose of 5 μg was associated with bone formation in 2/8 rabbits (FIG. 4A, B). No bone tissue was seen for the other LTA concentrations. The area of this newly formed bone was always less than 1% and the presence of bone was not significantly associated with one of the conditions.

### Effects of Inflammatory Stimuli on BMP-2 Induced Bone Formation

There were no signs of scaffolds degradation seen for the subcutaneously implanted constructs, which all contained BMP-2. These constructs demonstrated the presence of bone in various amounts in almost all groups. Only the condition loaded with the high dose (10 _{K}g) of LPS did not show any bone. In regions where bone tissue was found, fat tissue was also present in varying amounts. The rest of the BCP pore spaces were filled with fibrous tissue.

A relatively low dose of BMP-2 (1.5 μg) was chosen to discriminate the effects of the studied inflammatory stimuli. As a result, there was a large variation in the amount of bone in the samples of the control group (5.7±5.3 area %, FIG. 5B). In agreement with the histological observations, LPS was associated with a dose-dependent decrease in bone formation. Estimates based on mixed-model regression showed a 3.5-point drop in bone area% (p=0.010) for each 10-fold increase in the LPS concentration (FIG. 5C). TNF-α on the other hand, had a stimulatory effect on bone formation, which was also concentration-dependent. Constructs containing 10 ng TNF-α were associated with the most prominent bone formation seen in this study. In this group, the bone area % was doubled (13.6±9.9, p=0.005) compared to the control (5.7±5.3 area %). A higher concentration of TNF-α resulted in a reduction of bone formation. Although statistical analysis did not show a difference in the average amount of bone between the control group and the LTA-loaded groups, a borderline linear correlation existed between the LTA concentration and the bone area% (P=0.074) (FIG. 5C). For the lowest LTA concentration tested, a modest decrease in bone volume was seen, while an increase was found for the highest LTA concentration.

The fluorochromes revealed the dynamics of new bone formation in these subcutaneously implanted samples (FIG. 6). In the bone-containing samples, the 4-week label was located against the border of the BCP, indicating that this bone formed before 4 weeks implantation. The 8-week label was present throughout the bone indicating that mineralisation continued between week 4 and week 8. By gross examination, this label seemed to be more abundant in the BCP constructs loaded with TNF-α (FIG. 6, right panel). In these samples, the 4-week label was observed less frequently (FIG. 6, middle and lower panel). As these samples were associated with more bone after 12 weeks, it is plausible that the 8-week label was incorporated during remodelling of early-formed bone. The 11-week label was only seen sporadically, suggesting that there was less active bone mineralisation at this time point.

### Local Immune Response

The only immune response present at 12 weeks consisted of dense lymphoid cell clusters (FIG. 7A). Immunohistochemical staining revealed that approximately half of these cells had a T cell phenotype. The immune cell clusters did not contain granulocytes, indicating an absence of an acute inflammation at that time. After quantification of the lymphoid clusters in the different groups, it appeared that constructs loaded with LTA had the lowest average number of lymphoid clusters, while the opposite was found for LPS loaded constructs (FIG. 7B). The frequency of the lymphoid cell clusters found was comparable for both the subcutaneous and intramuscular samples. There was no apparent correlation between these two parameters when studying the number of lymphoid cell clusters and bone formation at the level of individual samples. Furthermore, by gross evaluation, there was also no clear evidence of co-localisation between the lymphoid clusters and bone tissue.

### Methods (FIGS. 8 & 9)

### Bacteria

The following bacteria were cultured to mid-log phase. E. coli (MG1656) and S. aureus (Wood 46) were cultured in LB medium at 37° C. H. influenzae (NCTC 8468) was cultured in BHI broth supplemented with X & V factors. M. marinum was cultured in medium at 30° C. B. cereus (ATCC 14579) was cultured in BHI medium at 30° C. The suspensions were centrifuged and washed extensively with ice cold PBS. A sample was taken for culture and CFU counting on blood agar (S. aureus, E.coli, B. cereus), chocolate agar supplemented with X&V factors (H. influenzae), or 7H10 agar (M. Marinum) plates. The bacteria were stored overnight in PBS at 4° C. The bacteria were then gamma-irradiated at 25 kGy (Isotron Nederland, Ede, The Netherlands). Their inactivation was confirmed by culture on agar plates. The bacteria were stored at −80° C. in PBS supplemented with 20% glycerol until the day of implantation.

### Sample Preparation

Biphasic calcium phosphate (BCP) blocks were made with dimensions of 6×6×3 mm (rabbits). These BCP blocks consisted of 20±5% β-tricalcium phosphate and 80±5% hydroxyapatite by weight, and had a total porosity of 75±5%. The BCP blocks were autoclaved at 121° C. for 30 min and dried at 60° C. The scaffolds were loaded with different concentrations of lipoteichoic acid (n=10) or whole inactivated bacteria [S. aureus (n=9), B. cereus (n=7), E. coli (n=7), H. influenzae (n=7), M. marinum (n=7)]. The scaffolds were implanted in subcutaneous or intramuscular pockets in the rabbits. In the subcutaneous location, the pro-inflammatory mediators were tested alone for the osteoinductive effect. In the intramuscular location, the pro-inflammatory were co-loaded with bone morphogenetic protein 2 to test the modulatory effect on BMP-2 osteoinduction.

### Sample Preparation

On the day of surgery, the mediators were diluted in PBS to a final concentration of 0.3-3 mg/ml LTA or 2×10⁵-2×10⁹ CFU/ml inactivated bacteria. In half of the samples, BMP-2 was added at a concentration of 21.5 g/ml. A volume of 70 μl was seeded onto the BCP blocks. Samples were stored at 4° C. until implantation.

### Animal Model

Female New Zealand White rabbits (2.5-3.0 kg, Charles River, L' Arbresle, France) were housed at the Central Laboratory Animal Institute, Utrecht University. The rabbits' daily diet consisted of 100 grams of pellet food (Stanrab, SDS, Essex, UK). Water was available ad libitum. After acclimatisation, surgery was performed under general anaesthesia. Rabbits received ketamine (15 mg/kg i.m.; Narketan®, Vétoquinol, 's-Hertogenbosch, The Netherlands) and glycopyrrolate (0.1 mg/kg i.m.; Robinul®, Riemser Arzneimittel A G, Greifswald, Germany) preoperatively, and medetomidine (0.25 mg/kg s.c.; Dexdomitor®, Orion Corporation, Espoo, Finland) perioperatively. Anaesthesia was reversed with atipamezole hydrochloride (0.5-1.0 mg/kg i.v., Atipam®, Eurovet Animal Health, Bladel, The Netherlands). Buprenorphine (0.03 mg/kg s.c.; Temgesic®, RB Pharmaceuticals Limited, Slough, UK) was given every 12 h for 2 days to relieve pain. After shaving and disinfecting the skin with 10% povidone-iodine, a midline incision was made to expose the paraspinal muscles. For implantation of BCP blocks, intramuscular pockets were created by blunt dissection. The pockets were closed with a non-resorbable suture (Prolene®, Ethicon, USA). For subcutaneous implantations, pockets were created through a 0.5 cm incision followed by blunt dissection. On day 56, the rabbits were euthanized with pentobarbital (i.v. Euthanimal®, Alfasan, Woerden, The Netherlands) under the same anaesthesia as described previously and BCP scaffolds were harvested.

### Analysis of Bone Formation

The material was fixed in 4% paraformaldehyde, dehydrated by an ethanol series and embedded in methyl methacrylate (MMA, Merck Millipore). Subsequently, 35 μm-thick sections were cut using a sawing microtome (Leica, Nusslochh, Germany) and stained with basic fuchsin and methylene blue. Two mid-sections were pseudo-coloured in Adobe Photoshop CS6 (Adobe Systems, San Jose, USA) to quantify the percentage of bone in the available pore space (bone area%). The mean value of two sections was used for further statistical analyses.

### Statistical Analyses

Results are shown as the mean±standard deviation. Statistics were performed using SPSS version 20.0 (IBM, Chicago, USA). Differences in bone area% were analysed using a linear mixed-model approach.

### Results (FIGS. 8 & 9)

### Effects of Inactivated Bacteria or Bacterial Antigen on Bone Formation Without BMP-2 and on BMP-2 Induced Bone Formation.

FIG. 8 shows that the bacteria and LTA induce very little to no bone formation at all when there is no BMP -2 is present. In control group consisting of BCP scaffolds alone, 3 out of 24 samples showed bone formation. In addition, in several samples that include inactivated bacteria bone formation was observed, with E. coli, M. marinum and S. aureus, 1/7, 1/7 and 2/9 of the samples respectively. However the amount of bone that was formed is very little (in all samples<1% bone area %) and therefore has little clinical relevance. In addition, in all other samples containing various concentrations of inactivated bacteria or LTA no bone formation was observed. These results show that inactivated bacteria or bacterial antigen have little to no effect on bone formation in ectopic sites.

In succession to the previous experiment we tested the effect of inactivated bacteria on BMP-2 induced bone formation, FIG. 9. The control group consisting of BCP scaffolds containing BMP-2 was compared to BCP scaffolds containing BMP-2 and various concentrations of inactivated bacteria. The results show that when using bacteria in combination with BMP-2 the % of bone area is increased at a higher level, than when only BMP-2 is used. This indicates that inactivated bacteria have a synergistic effect on the bone formation promoting effect induced by BMP-2. However, it also seems that a too high concentration will have a negative impact on the induced bone formation.

### Methods (Table 3)

### Bacteria

S. aureus (Wood 46) were cultured to mid-log phase in LB medium. The bacteria were stored overnight in PBS at 4° C. and gamma-irradiated at 25 kGy (Isotron Nederland, Ede, The Netherlands). Their inactivation was confirmed by culture on agar plates. The bacteria were stored at −80° C. in PBS supplemented with 20% glycerol until the day of implantation.

### Sample Preparation

Biphasic calcium phosphate (BCP) blocks were made with dimensions of 3.5×3.5×3 mm. These BCP blocks consisted of 20±5% β-tricalcium phosphate and 80±5% hydroxyapatite by weight, and had a total porosity of 75±5% (Yuan et al., 2002). The BCP blocks were autoclaved at 121° C. for 30 min and dried at 60° C. A volume of 35 μl was seeded onto the BCP blocks with a final concentration of bacteria of 2×10⁶-2×10¹⁰ CFU/ml. In half of the samples, BMP-2 was added at a final concentration of 15 μg/ml. Samples were stored at 4° C. until implantation.

### Animal Model

8 male Fischer rats (14 weeks old, 300-350 g, F344/IcoCrl, Charles River) were used for the experiment. All animals were housed at the Central Laboratory Animal Institute, Utrecht University. They were allowed to acclimatize for at least two weeks before the surgery. Food and water were given ad libitum. The rats received 3% isoflurane during surgery. Animals were given pain relief preoperatively, and postoperatively every 8 hours for 2 days with Buprenorphine (0.03 mg/kg s.c.; Temgesic®, RB Pharmaceuticals Limited, Slough, UK). After shaving and disinfecting the skin with povidone-iodine (Betadine), a midline incision was made to expose the paraspinal muscles. Intramuscular pockets were by blunt dissection for implantation of the BCP blocks. Pockets were closed with a non-resorbable suture (Prolene®, Ethicon, USA), followed by closure of the skin (Monocryl®, Ethicon). Subcuteanous pockets were made in the same fashion. The rats were killed with CO₂, 8 weeks after the second surgery and the BCP blocks were explanted.

### Bone Histomorphometry

The material was fixed in 4% paraformaldehyde, dehydrated by an ethanol series and embedded in methyl methacrylate (MMA, Merck Millipore). Subsequently, 35 μm-thick sections were cut using a sawing microtome (Leica, Nusslochh, Germany) and stained with basic fuchsin and methylene blue. The samples were completely sectioned and scored for the presence of bone.

### Results (Table 3)

One rat died immediately after surgery. There were no complications in the other rats.

In the empty BCP samples, no new bone formation was observed (Table 3). Furthermore, the loading of inactivated bacteria onto the BCP did not results in osteoinduction. In contrast, the inactivated bacteria had a stimulatory effect on number of bone-containing samples in BMP-2 loaded BCP constructs. The highest concentration of bacteria (2.5×10¹⁰ CFU/ml) inhibited new bone formation.

TABLE 3
Ectopic bone formation in biphasic calcium
phosphate (BCP) blocks implanted in rats.

| | Implantation | No. rats |
|---|---|---|
| BCP loading | location | with bone |
| 15 μg/ml BMP-2 | Subcutaneous | 1/7 |
| 15 μg/ml BMP + 2.5 × 10⁶ | Subcutaneous | 3/7 |
| inactivated S. aureus | | |
| 15 μg/ml BMP + 2.5 × 10⁸ | Subcutaneous | 4/7 |
| inactivated S. aureus | | |
| 15 μg/ml BMP + 2.5 × 10¹⁰ | Subcutaneous | 0/4 |
| inactivated S. aureus | | |
| Empty | Intramuscular | 0/7 |
| 2.5 × 10⁶ inactivated S. aureus | Intramuscular | 0/7 |
| 2.5 × 10⁸ inactivated S. aureus | Intramuscular | 0/7 |
| 2.5 × 10¹⁰ inactivated S. aureus | Intramuscular | 0/7 |

1. A composition for use in the treatment of an individual suffering from a condition necessitating new bone formation, said composition comprising at least one immunomodulation agent and at least one bone formation promoting agent, wherein the composition induces a local inflammation.

2. The composition for use according to claim 1, wherein the composition provides bone tissue generation at an ectopic location.

3. The composition for use according to claim 1, wherein the composition provides bone tissue generation at the site of the condition necessitating new bone formation.

4. The composition for use according to claim 1, wherein the at least one immunomodulation agent is selected from the group consisting of inactivated bacteria, bacteria, attenuated bacteria, modified bacteria, bacterial antigens, bacterial cell wall extracts, cytokines, viral antigens, inactivated microorganisms, prostaglandins and pro-inflammatory lipoproteins, preferably inactivated bacteria.

5. The composition for use according to claim 1, wherein the at least one bone formation promoting agent is selected from the group consisting of bone-stimulating growth factors, bone-stimulating bisphosphonates and bone antiresorptive factors.

6. The composition for use according to claim 5, wherein the bone-stimulating growth factors are selected from the group consisting of bone morphogenetic proteins (BMPs), BMP-2, BMP-1, BMP-3, BMP-4, BMP-5, BMP-6, BMP-7, BMP-8a, BMP-8b, BMP-9, BMP-10, BMP-15, transforming growth factor (TGF), fibroblast growth factor (FGF), vascular endothelial growth factor (VEGF), insulin like growth factor (IGF), and heterodimers, fusion proteins and/or isoforms thereof, preferably BMP-2.

7. The composition for use according to claim 5, wherein the bone-stimulating bisphosphonates are selected from the group consisting of alendronate, pamidronate, clodronate and zoledronate.

8. The composition for use according to claim 5, wherein the bone antiresorptive factors are selected from the group consisting of alendronate, pamidronate, clodronate, zoledronate, parathyroid hormone, osteoprotegerin, or SERMs (selective estrogen receptor modulators).

9. The composition for use according to claim 1, wherein the at least one immunomodulation agent is bacteria selected from the group of the genus of Staphylococcus, Escherichia, Mycobacterium, Bacillus and Haemophilus.

10. The composition for use according to claim 1, wherein the at least one immunomodulation agent is a bacterial antigen selected from the group consisting of lipoteichoic acid (LTA), lipoproteins, bacterial cell wall extracts, polysaccharide, lipopolysaccharide (LPS), glycolipids, or peptidoglycans.

11. The composition for use according to claim 1, wherein the at least one immunomodulation agent is a pro-inflammatory cytokine selected from the group consisting of TNF-α, IL-17, IL-1, IL-6, IFN-γ and heterodimers, fusion proteins and/or isoforms thereof.

12. The composition for use according to claim 1, wherein said at least one immunomodulation agent is TNF-α and said at least one bone formation promoting agent is BMP-2.

13. The composition for use according to claim 1, wherein the at least one bone formation promoting agent is BMP at a concentration of between 0.1 μg/ml and 5 mg/ml.

14. The composition for use according to claim 1, wherein the at least one immunomodulation agent is bacteria at a concentration of between 10² to 10¹² bacteria/ml.

15. The composition for use according to claim 1, wherein the at least one immunomodulation agent is bacterial antigen at a concentration of between 0.05 to 5 μg/ml.

16. The composition for use according to claim 1, wherein the at least one immunomodulation agent is a cytokine at a concentration of between 0.1 to 1000 ng/ml.

17. The composition for use according claim 1, wherein the condition necessitating new bone formation is selected from the group consisting of large bone defects, segmental bone defects, structural bone defects, bone fractures, non-unions of bone fractures, fusion of joints including spinal fusions, and osteoporosis.

18. A medicament comprising a composition as defined in claim 1.

19. An osteoconductive carrier provided with a composition as defined in claim 1, wherein said carrier is comprised of collagen, hydrogel, calcium hydroxyapatite, calcium phosphate, calcium silicates, calcium sulphates, bioglass, titanium, tantalum and/or stainless steel.

20. A method for the treatment of an individual suffering from a condition necessitating new bone formation, the method comprising administering to said individual a composition as defined in claim 1.

21. The method according to claim 20, wherein said composition is administered via a local injection or via a carrier that is comprised of collagen, hydrogel, calcium hydroxyapatite, calcium phosphate, calcium silicates, calcium sulphates, bioglass, titanium, tantalum and/or stainless steel and is implanted in said individual.

22. (canceled)
